(19) United States
(12) Patent Application Publication
(10) Pub. No.: US 2025/0134722 A1
(43) Pub. Date: May 1, 2025

(54) A MEDICAL DRESSING

(71) Applicant: Mölnlycke Health Care AB, Göteborg (SE)

(72) Inventors: Conny Jakobsson, Lerum (SE); Daniel Melin, Mölnlycke (SE); Christian Wathne, Mölnlycke (SE)

(21) Appl. No.: 18/692,054

(22) PCT Filed: Sep. 28, 2021
(86) PCT No.: PCT/EP2021/076643

§ 371 Date: Mar. 14, 2024

(51) Int. Cl. A61F 13/0203; A61F 13/02

(52) U.S. Cl. CPC A61F 13/0226; A61F 13/0259

(57) ABSTRACT

Described is a medical dressing that has a first and a second release liner, wherein the first release liner includes at least one marking. The marking(s) may indicate how to cut, fold, use or apply the dressing. Also described is a kit that includes the described a medical dressing.

### TECHNICAL FIELD

The present disclosure generally relates to a medical dressing comprising a first and a second release liner, wherein the first release liner comprises at least one marking. The marking may indicate how to cut, fold, use or apply the dressing. The present disclosure also relates to a kit comprising such a medical dressing.

### BACKGROUND

Adhesive medical dressings are frequently used in wound care, both for the purpose of treating wounds and scars and for the purpose of preventing these from occurring in the first place.

Adhesive medical dressings typically comprise an adhesive wound contact layer arranged to contact the skin or wound of a patient, as well as a top layer, often referred to as a backing layer. In many cases, particularly when the dressing is to be applied to moderate or highly exuding wounds, the dressing further comprises a wound pad arranged between the backing layer and the adhesive wound contact layer. To protect the dressing from contamination, a release liner may be applied to the adhesive wound contact layer.

In a care facility or a hospital, medical dressings are typically applied to a patient by caregivers or medical staff, i.e. not by the patients themselves. In many situations, the caregivers and staff may face difficulties in how to properly use the dressing or how to properly apply the dressing to the patient. While instructions relating to a correct use and application of the dressing may be provided in an instruction manual (“Instructions for use”, IFU), inserted into the multi-pack in which the individual dressings are stored, such IFUs are generally not used or present in the actual care situation.

Many times, critical care scenarios arise in a hospital, and such scenarios are often associated with a sense of urgency in the application of a dressing to a patient. One example of such a situation is where a CPAP mask, a tracheostomy tubing, an oxygen tubing or any other type of life sustaining medical device is to be used in conjunction with the dressing. Accordingly, there is limited time for the caregivers to apply the dressings correctly, which may be experienced as stressful. Furthermore, the risk of applying the dressing incorrectly to the patient is enhanced.

In view of this, it would be desired to communicate to the caregivers or medical staff proper use or application of the dressing in a more direct, “on site” and “in situation” manner. Accordingly, there is a need to provide a facilitated means to guide the medical staff to a correct use and application of a medical dressing in a communicative, intuitive, and clear manner. Such means should facilitate the burden for caregivers and staff dealing with ulcers in critical care scenarios.

### SUMMARY

In view of the above-mentioned problems, it is an object of the present disclosure to provide improvements with respect to relieving the stress for caregivers in various care situations and providing a facilitated, clear and communicative means to guide the staff and caregivers to a correct use of the product.

According to a first aspect, there is provided a medical dressing comprising a backing layer, an adhesive skin contact layer and a first release liner detachably attached to the adhesive skin contact layer; the dressing further comprising a second release liner arranged between the adhesive skin contact layer and the first release liner, wherein the surface area of the second release liner is smaller than the surface area of the first release liner, wherein the first release liner is co-extensive with the backing layer and the adhesive skin contact layer and wherein the first release liner comprises at least one marking.

The present disclosure is based on the realization that the provision of a first release liner being co-extensive with the adhesive layer and with the backing layer facilitates the provision of markings onto the dressing. The markings provided on the first release liner may guide the caregivers and medical staff to a correct use or application of the dressing.

A co-extensive release liner portion allows for markings to be provided at the peripheral edges of the first release liner. Such edge markings may e.g. be useful to indicate that the dressing should be cut or folded. Furthermore, if the markings are provided by means of a printing process, the printing process is greatly facilitated by means of a co-extensive release liner.

The at least one marking may indicate how to cut, fold, use or apply the dressing.

Accordingly, the caregiver and medical staff is offered direct, understandable, and clear guidance in how to properly handle and apply the dressing. This is particularly advantageous in scenarios associated with a sense of urgency. Such situations may e.g. occur when a life-sustaining medical device, such as a CPAP mask, a tracheostomy or an oxygen tubing must be applied to the patient, and the area underneath the medical device should be protected from undesired pressure points resulting from the medical device.

The medical dressing of the present disclosure saves time and reduces the stress and strain among the medical staff in such care scenarios and situations.

If the markings provided on the first release liner indicate that the dressing should be cut or folded, the markings typically extend from one peripheral edge to another peripheral edge of the first release liner. The co-extensive first release liner thus facilitates the provision of such edge markings.

Accordingly, in embodiments, the first release liner is defined by a central portion and an edge portion surrounding the central portion, wherein the central portion constitutes from 70 to 95% of the surface area of the first release liner, wherein the at least one marking, is arranged in at least the edge portion of the first release liner.

In exemplary embodiments, the at least one marking, is formed by a print pattern, a weld pattern, a compressed pattern, an embossed pattern, a material deposition pattern, or combinations thereof.

Any pattern that allows the caregiver to visually recognize and follow the guidance provided by the at least one marking may be utilized.

Typically, the at least one marking is printed and/or embossed on the first release liner.

The printed markings are easily recognizable on the first release liner.

The embossed markings may be recognized by touch, and may e.g. facilitate cutting of the dressing due to the difference in tactility (in cases where cutting is a desired action).

In embodiments, the first release liner and the second release liner are formed from different materials.

This is particularly beneficial with a medical dressing of the present disclosure. A conventional release liner is typically formed from a first folded release liner portion and a second, larger release liner portion overlapping and extending slightly beyond the folded first release liner portion. The slight overlap between the folded release and the overlying release liner forms a “tab” that the wearer or caregiver can grasp to facilitate removal.

The first release liner of the present disclosure may be somewhat more difficult to grasp than a conventional release liner since it is co-extensive with the adhesive skin contact layer. The difficulties in grasping and removing the first release liner from the second release liner are particularly enhanced if the first and the second release liners are formed from the same materials. During manufacturing of the dressing, and particularly during assembly of the dressing components, the first and the second release liners tend to adhere to each other, particularly at the interface between the first and the second release liner, which, in the present case, is relatively large. One reason for such undesired adherence may be that heat generated from friction during assembly of the dressing components can cause the release liners to become partially welded or fused together. Hence, different materials with different melting points are advantageously utilized for the first and the second release liners. This facilitates the removal of the first release liner from the underlying second release liner (and from the dressing).

In exemplary embodiments, the first release liner comprises a first polymeric film, and wherein the second release liner comprises a second polymeric film, wherein the first polymeric film is different from the second polymeric film.

A polymeric film allows for printing, welding, compressing and/or embossing the markings onto the first release liner. The first polymeric film is different from the second polymeric film for the reasons explained hereinabove.

In exemplary embodiments, the first and/or the second polymeric film is selected from a polyethylene film, a polypropylene film and a polyurethane film.

Preferably, the first polymeric film is a polyethylene film and the second polymeric film is a polypropylene film.

These polymeric films have different melting points and undesired fusion between the first and the second release liner is prevented during assembly or manufacturing of the medical dressing

In exemplary embodiments, the dressing comprises a pad arranged between the backing layer and the adhesive skin contact layer. The pad may be co-extensive with the adhesive skin contact layer and the backing layer.

The pad is preferably absorbent. This is to improve the handling of body fluids, e.g. blood, pus or sweat.

In embodiments, the pad comprises a pressure-relieving material. Preferably the pad comprises a material that allows for the dressing to be cut through.

In cases where the dressing is to be used in conjunction with a medical device, a pressure-relieving material serves to protect the patient's skin from the load and shear imparted by an overlying medical device. Accordingly, medical device related pressure ulcers (MDRPU) may be prevented.

In embodiments, the dressing comprises a longitudinal center line dividing the first release liner into a first portion and a second portion; the first and the second portion being symmetric about the longitudinal center line, wherein the first portion comprises a first set of markings, and wherein the second portion comprises a second set of markings, and wherein the first set of markings is different from the second set of markings.

The first set of markings may indicate a correct use, application, cutting or folding of the dressing that is adapted for a first specific care situation. The second set of markings may indicate a correct use, application, cutting or folding of the dressing that is adapted for a second, different care situation.

In embodiments, the first set of markings comprises a first set of guiding lines indicating how to cut the dressing into a first set of dressing pieces, and wherein the second set of markings comprises a second set of guiding lines indicating how to cut the dressing into a second set of dressing pieces.

The first and/or the second set of guiding lines may be provided to guide a caregiver to cut through the guiding lines. Thereby, a plurality of dressing pieces may be obtained, wherein each dressing piece has a shape configured to mitigate pressure points from at least one medical device. Each of the cut-out dressing pieces is adapted to fit a particular facial area and to fit with a specific medical device.

In exemplary embodiments, the longitudinal center line is a folding line, and wherein the dressing can be arranged in a first, flat configuration, and in a second, folded configuration about the folding line, wherein the first set of guiding lines indicate how to cut the dressing in the folded configuration, and wherein the second set of guiding lines indicate how to cut the dressing in the folded configuration.

The provision of a folding line, about which the dressing is folded when the dressing is cut, allows for a plurality of different guiding lines to fit on a small dressing area. Accordingly, a variety of different dressing shapes may be obtained despite the limited space on the dressing. This allows for an optimized utilization of the dressing material. Less material is needed, and material waste resulting from dressing the patient with various tape pieces in an arbitrary manner (which is the conventional scenario in a hospital setting), is considerably reduced.

The dressing pieces of the first and second sets of dressing pieces may have a shape adapted to fit with at least one medical device.

For example, the dressing pieces of the first set of dressing pieces may be adapted for a first medical device and the dressing pieces of the second set of dressing pieces may be adapted for a second medical device.

Accordingly, the caregiver may selectively choose to cut in the first or second portion depending on the specific need and specific medical device that is to be applied to the patient. For example, if an oxygen tubing is to be used, the first set of guiding lines on the first portion of the dressing may indicate how to cut the dressing into the dressing shapes adapted for such oxygen tubing. Furthermore, if a CPAP mask or any other medical device is to be used, the second set of guiding lines on the second portion may be adapted for the provision of dressing shapes that fit the CPAP mask (or vice versa).

To date, the medical personnel may apply various types of tapes and padded material sheets underneath a specific medical device. For example, the medical personnel may utilize various tape pieces and dress the facial areas exposed to a medical device. Alternatively, the medical personnel may start from a larger tape or padded material sheet and cut the sheet into a shape that fits with the medical device to be utilized for a specific patient and a specific care scenario.

However, in urgent and critical situations in a hospital, the staff must typically quickly apply the medical device to the patient. Accordingly, there is a limited time to “dress” the dermal area underneath the medical device. Instead of guessing where and how the tape or material sheet is to be applied, and potentially cutting the tape or material sheet into an incorrect shape, the caregiver is offered “on site” assistance, which allows for the specific requirements and needs of the patient to be met. This saves time and reduces stress associated with such critical care scenarios for the staff dealing with medical devices and pressure ulcers resulting from such devices.

In exemplary embodiments, the at least one marking, comprises a fold marking indicating that the dressing should be folded prior to cutting.

Accordingly, the staff or caregivers are directed to folding the dressing prior to cutting the dressing into pieces.

In exemplary embodiments, the at least one marking is a printed marking formed by a colored ink.

The colored ink may have a hydrophobic character to prevent the ink from dissolving in contact with body fluids.

In exemplary embodiments, the first portion of the first release liner has a texture, color or shade that differs from the texture, color or shade of the second portion of the first release liner.

The difference in texture, color and/or shade of the first, and the second portion, respectively, may be provided to emphasize that the dressing should be folded and/or to emphasize that the respective portion yield different dressing shapes tailored for different purposes, e.g. tailored for different types of medical devices (depending on which portion the caregiver chooses to cut).

The difference in color or shade may also be regarded as visually appealing to the viewer.

In exemplary embodiments, the first portion of the first release liner is white or transparent and wherein the second portion is colored.

Accordingly, a distinct difference between the portions is provided.

In exemplary embodiments, the colored second portion of the first release liner and the at least one printed marking are formed by the same colored ink, wherein the shade of the colored second portion is lighter than the shade of the at least one printed marking.

By utilizing the same colored ink for printing both the second portion and the at least one marking, the manufacturing of the first release liner is significantly improved. By varying the contrast and shade of the color of the ink, a distinct and visually appealing contrast is created between the marking(s) and the colored second portion. The shade of the printed markings may also vary individually between the markings provided on the first release liner. For example, the first set of markings may have a different shade than the shade of the second set of markings.

In exemplary embodiments, the first release liner has a surface tension that is higher than the surface tension of the colored ink.

This allows for the printed markings to adhere better to the first release liner. Accordingly, printing is facilitated and the printed markings (guiding lines, colored portions etc.) will have an improved adherence to the first release liner.

In exemplary embodiments, the first release liner has a lateral (x) extension defining the width of the first release liner and a longitudinal (y) extension defining the length of the first release liner, wherein the width of the second release liner corresponds to the width of the first release liner, and wherein the length of the second release liner corresponds to from 20 to 60%, preferably from 30 to 40% of the length of the first release liner.

Accordingly, the second release liner extends in a transverse direction with respect to the folding line, when present. This arrangement is beneficial, since after the dressing pieces have been cut into their respective shaped dressing pieces, the majority of the dressing pieces will comprise at least a portion of the second, smaller release liner. Accordingly, the removal of the first release liner overlying each of the cut-out dressing pieces will be facilitated.

According to a second aspect, there is provided a kit comprising a medical dressing as defined hereinbefore and a medical device.

Further features of, and advantages with, the present disclosure will become apparent when studying the appended claims and the following description. The skilled addressee realizes that different features of the present disclosure may be combined to create embodiments other than those described in the following, without departing from the scope of the present disclosure.

### BRIEF DESCRIPTION OF THE DRAWINGS

The various aspects of the present disclosure, including its particular features and advantages, will be readily understood from the following detailed description and the accompanying drawings, in which:

FIG. 1A is a split-view illustrating a dressing according to an exemplary embodiment of the present disclosure.

FIG. 1B illustrates a top-view of a dressing according to an exemplary embodiment of the present disclosure, illustrating the markings provided on the first release liner.

FIG. 1C illustrates the dressing of FIG. 1A in a folded configuration.

FIG. 2A illustrates the shaped dressing pieces formed when the dressing has been cut along the markings provided on the first release liner; i.e. along the first guiding line of the first set of guiding lines in the folded configuration, according to an exemplary embodiment of the present disclosure.

FIG. 2B illustrates the shaped dressing pieces formed when the dressing has been cut along the markings provided on the first release liner; i.e. along the first and the second guiding lines of the first set of guiding lines in the folded configuration, according to an exemplary embodiment of the present disclosure.

FIG. 2C illustrates a front-view of a patient wearing the dressing pieces of FIG. 2B.

FIG. 2D illustrates a side-view of a patient wearing the dressing pieces of FIG. 2B.

FIG. 3A illustrates the shaped dressing pieces formed when the dressing has been cut along the markings provided on the first release liner; i.e. along the third guiding line of the first set of guiding lines in the folded configuration, and along the folding line, according to an exemplary embodiment of the present disclosure

FIGS. 3B and 3C illustrate side views of a patient wearing the dressing pieces of FIG. 3B.

FIG. 4A illustrates the shaped dressing pieces formed when the dressing has been cut along the markings provided on the first release liner; i.e. along the first and second guiding lines of the second set of guiding lines in the folded configuration, according to an exemplary embodiment of the present disclosure FIG. 4B illustrates a front-view of a patient wearing the dressing pieces of FIG. 4A.

FIG. 4C illustrates a side-view of a patient wearing the dressing pieces of FIG. 4A.

FIG. 4D illustrates the shaped dressing pieces formed when the dressing has been cut along the markings provided on the first release liner; i.e. along first, second and third guiding lines of the second set of guiding lines in the folded configuration, according to an exemplary embodiment of the present disclosure.

FIG. 4E illustrates a front-view of a patient wearing the dressing pieces of FIG. 4D.

### DETAILED DESCRIPTION

The present disclosure will now be described more fully hereinafter with reference to the accompanying drawings, in which currently preferred embodiments of the present disclosure are shown. The present disclosure may, however, be embodied in many different forms and should not be construed as limited to the embodiments set forth herein; rather, these embodiments are provided for thoroughness and completeness, and fully convey the scope of the present disclosure to the skilled person. Like reference characters refer to like elements throughout.

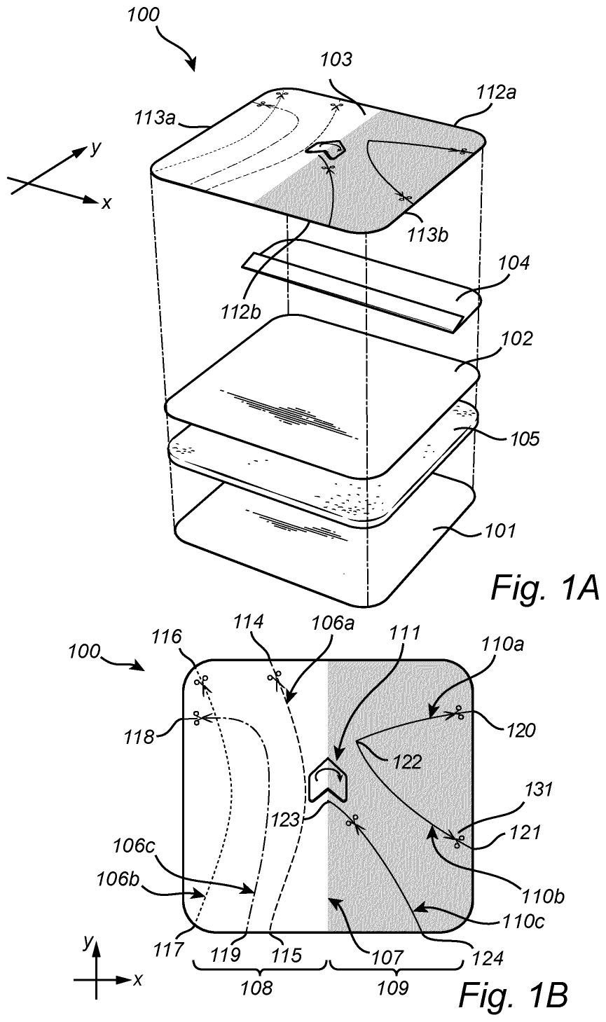

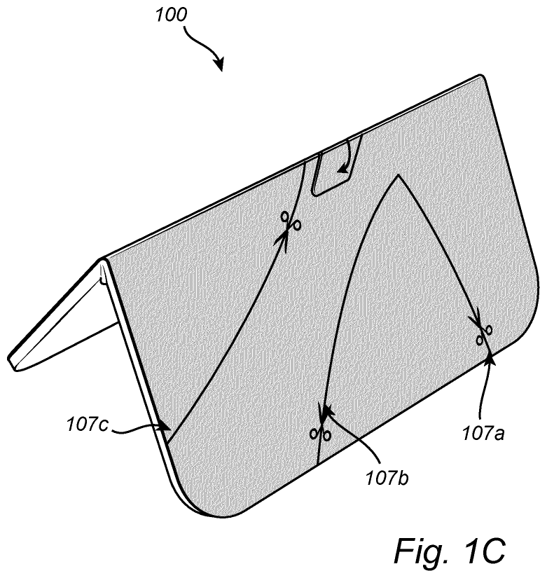

With reference to FIG. 1A, a medical dressing 100 according to the present disclosure is conceptually illustrated. The dressing 100 comprises a backing layer 101, an adhesive skin contact layer 102 and a first release liner 103 detachably attached to the adhesive skin contact layer 102; the dressing 100 further comprising a second release liner 104 arranged between the adhesive skin contact layer 102 and the first release liner 103, wherein the surface area of the second release liner 104 is smaller than the surface area of the first release liner 103, wherein the first release liner 103 is co-extensive with the backing layer 101 and the adhesive skin contact layer 102 and wherein the first release liner 103 comprises at least one marking (106a-c, 110a-c, 111).

The first release liner 103 is co-extensive in width and in length with the backing layer 101 and the adhesive skin contact layer 102. Accordingly, the surface area of the first release liner 103 corresponds to the surface area of the backing layer 101 and the adhesive skin contact layer 102. In embodiments where the dressing comprises a pad, the first release liner may be co-extensive with the pad.

The second release liner 104 may be configured to cover from 20 to 50%, e.g. from 30 to 40% of the surface area of the adhesive skin contact layer. The first release liner 103 may be configured to cover from 50 to 80% e.g. from 60 to 70% of the surface are of the adhesive skin contact layer. Accordingly, the area of the first release liner 103 overlapping the second release liner 104 is from 20 to 50%, e.g. from 30 to 40%.

The second release liner is typically folded (see FIG. 1A). The folded portion forms a gripping tab that the applicator can grasp after the first release liner has been removed or displaced from its covering position.

As used herein, the term “at least one marking” means any type of marking preferably indicating a correct use or application of the dressing. For example, the at least one marking may indicate how to cut, fold, use or apply the dressing. The marking(s) is/are visually recognizable to the caregiver. The marking(s) may be formed by a print pattern, a weld pattern, a compressed pattern, an embossed pattern, a material deposition pattern, or combinations thereof. Typically, the marking(s) is/are printed or embossed. If the marking(s) is/are embossed, the marking(s) is/are also recognizable by touching. The markings in FIGS. 1A-1C are printed on the first release liner. The markings may alternatively, or additionally, be embossed. The first release liner has a first surface facing the adhesive skin contact layer and a second opposing surface. The second surface faces the viewer. Typically, when the at least one marking is formed by printing, the at least one marking is printed onto the second surface of the first release liner.

The first release liner 103 may be defined by a central portion and an edge portion surrounding the central portion, wherein the central portion constitutes from 70 to 95% of the surface area of the first release liner 103, wherein the at least one marking (106a-c, 110a-c, 111) is arranged in at least the edge portion of the first release liner 103.

As can be seen, the markings illustrated in FIGS. 1A and 1B are arranged to extend from the peripheral edges (112a-b, 113a-b) of the first release liner 103. This is achievable due to the co-extensive character of the first release liner of the medical dressing of the present disclosure. In some cases, there may be deviations from this due to tolerances in the manufacturing process. Either way, the at least one marking is preferably not arranged further away from a peripheral edge (112a-b, 113a-b) than 5 mm. preferably 3 mm.

The first release liner 103 and the second release liner 104 may be formed from different materials.

Typically, the first release liner 103 comprises a first polymeric film, and wherein the second release liner 104 comprises a second polymeric film, wherein the first polymeric film is different from the second polymeric film.

A polymeric film is beneficial since it allows for printing, welding, compressing and/or embossing the markings onto the first release liner.

The different materials; i.e. polymeric films of the first and second release liners prevents undesired fusion or adherence between the first and second release liner in the area where the first release liner overlaps with the second release liner. Accordingly, removal of the first release liner from the second release liner (and from the dressing) is facilitated.

The first and/or the second polymeric film may be selected from a polyethylene film, a polypropylene film and a polyurethane film.

The first and/or the second release liner may consist of the polymeric film or may be a laminate comprising the polymeric film.

The thickness of the first and/or the second polymeric film is typically in the range of from 20 to 150 μm, e.g. from 40 to 120 μm, e.g. from 70 to 100 μm.

The first release liner preferably comprises a polyethylene or a polypropylene film having a thickness in the range of from 40 to 140 μm, e.g. from 70 to 100 μm.

Suitably, the first polymeric film is a polyethylene film and the second polymeric film is a polypropylene film.

As illustrated in FIG. 1A, the dressing 100 may comprise a pad 105 arranged between the backing layer 101 and the adhesive skin contact layer 102. The pad 105 is co-extensive with the backing layer 101 and the adhesive skin contact layer 102.

The pad is preferably absorbent and comprises a pressure-relieving material. If the dressing is to be used in conjunction with a medical device; i.e. for the purpose of alleviating medical device related pressure ulcers (MDRPU), an improved pressure-relieving effect may thus be obtained, and undesired scarfing caused by the medical device is avoided.

The pad is not limited to a specific material, but preferably allows for the dressing to be cut through and yielding a pressure-relieving effect.

For example, the pad may comprise a hydrophilic and/or absorbent foam. Preferably, the pad comprises a polyurethane foam.

The pad 105 may comprise one or more layers. If the pad comprises a plurality of pad-forming layers, the pad-forming layers may be laminated or attached to each other to facilitate cutting through the dressing. Preferably, the pad is void of superabsorbent particles or gel forming fibers, which may “leak” from the dressing during cutting.

The thickness of the pad may be in the range of from 1 to 5 mm, e.g. from 2 to 3 mm. If the thickness of the pad is too large, it may become difficult to fold and/or cut through the dressing.

As used herein, the term “adhesive skin contact layer” means a layer configured to detachably adhere the dressing to a dermal surface. In other words, the adhesive skin contact layer is configured to contact the skin of a wearer.

Preferably, the adhesive skin contact layer comprises a silicone-based adhesive. A silicone-based adhesive is gentle to the skin and may be removed from the skin without causing any trauma. For example, the adhesive skin contact layer may comprise a silicone gel. The silicone gel may be provided as a coating on the pad.

The adhesive skin contact layer may comprise one or more sub-layers. For example, the adhesive skin contact layer may comprise a polymeric film and an adhesive silicone gel layer, wherein the adhesive silicone gel layer is arranged to contact the skin.

In embodiments where the dressing comprises a pad, the backing layer 101 may be adhesively attached to the pad. Alternatively, the backing layer 101 may be laminated to the pad. For example, heat lamination may be utilized to apply the backing layer 101 to the pad

As illustrated in FIGS. 1A and 1B, the first release liner 103 has a lateral (x) extension and a longitudinal (y) extension, wherein the dressing comprises a longitudinal center line dividing the first release liner into a first portion 108 and a second portion 109; the first 108 and the second 109 portion being symmetric about the longitudinal center line, wherein the first portion 108 comprises a first set of markings 106a-c, and wherein the second portion 109 comprises a second set of markings 110a-c, and wherein the first set of markings 106a-c is different from the second set of markings 110a-c.

The first set of markings may indicate a correct use, application, cutting or folding of the dressing that is adapted for a first specific care situation. The second set of markings may indicate a correct use, application, cutting or folding of the dressing that is adapted for a second, different care situation.

As best illustrated in FIG. 1B, the first set of markings may comprise a first set of guiding lines 106a-c indicating how to cut the dressing into a first set of dressing pieces, and wherein the second set of markings 110a-c comprises a second set of guiding lines indicating how to cut the dressing into a second set of dressing pieces.

As used herein the term “first and/or second sets of guiding lines” means a plurality of lines that are visually recognizable to the caregiver. The first and/or the second sets of guiding lines may be formed by a print pattern, a weld pattern, a compressed pattern, an embossed pattern, a material deposition pattern, or combinations thereof. Typically, the first and/or second sets of guiding lines are printed or embossed. If the guiding lines are embossed, the guiding lines are also recognizable by touching. If the guiding lines are printed on the first release liner, the printed guiding lines may represent printed solid, continuous, dotted, or discontinuous lines. The printed guiding line may also be associated with scissor markings to further emphasize that the guiding lines should be cut.

A first or a second “set” of guiding lines typically comprises at least two, e.g. at least three guiding lines.

A first or a second “set” of dressing pieces typically means at least three dressing pieces. The dressing pieces may be applied to any dermal area underlying a medical device. Typically, the dressing pieces are applied to a facial area of the patient.

The dressing pieces obtained after cutting the first and/or the second set of guiding lines may have a shape configured to mitigate pressure points from at least one medical device. Each of the cut-out dressing pieces may be adapted to fit a particular facial area and to fit with a specific medical device.

The guiding lines of the first set of guiding lines may be adapted to yield dressing pieces adapted for a first facial area, and the second set of guiding lines may be adapted to yield dressing pieces adapted for a second facial area.

The term facial area is to be interpreted broadly. The “facial area” includes all parts of the patient's face, such as the cheeks, nose, nose ridge, forehead, the area between the mouth and the nose, and also includes the chin, neck and the area behind the ears.

In embodiments, the longitudinal center line is a folding line 107, and wherein the dressing can be arranged in a first, flat configuration, and in a second, folded configuration about the folding line 107, wherein the first set of guiding lines 106a-c indicate how to cut the dressing in the folded configuration, and wherein the second set of guiding lines 110a-c indicate how to cut the dressing in the folded configuration.

As used herein, the term “folding line” means a centrally disposed line dividing the dressing into a first and a second portion, respectively, and which indicates to the caregiver that the dressing should be folded. The folding line may be formed by a print pattern, a weld pattern, a compressed pattern, an embossed pattern, a material deposition pattern, or combinations thereof. The folding line may be a printed and/or embossed folding line. The folding line may also be formed from a difference in texture, color or shade of the first and the second portion of the first release liner. Accordingly, the intersection between the first and the second portion may represent the “folding line” (in embodiments where the texture/color/shade of the first portion is different from the texture/color/shade of the second portion, see FIGS. 1A-1C).

As illustrated in FIGS. 1A and 1B, the first set of guiding lines 106a-c is different from the second set of guiding lines 110a-c.

In this context, “different” means that the first set of guiding lines yield a first set of dressing pieces being different from the dressing pieces obtained from the second set of guiding lines.

The first set of guiding lines may also be different from the second set of guiding lines in the sense of the character of the guiding lines. As illustrated in FIGS. 1A and 1B, the first set of printed guiding lines 106a-c comprises discontinuous or dotted lines. The second set of printed guiding lines 110a-c comprises continuous or bold lines. This is to further emphasize the difference between the first and the second set of guiding lines.

The caregiver may decide to cut the first set of guiding lines or the second sets of guiding lines depending on the specific care situation, the specific dermal or facial area to be protected, and the specific medical device to be used. A plurality of different dressing shapes may be obtained adapted to fit with one or more specific medical device(s). The guiding lines provided on the first release liner guides the caregiver to first fold the dressing (see FIG. 1C), and then cut the dressing in the folded configuration by following the first, and/or the second set of guiding lines disposed on the first release liner.

As mentioned hereinbefore, the plurality of dressing pieces are adapted to be used in conjunction with at least one medical device. The first set of guiding lines 106a-c may be adapted for a first medical device. The second set of guiding lines 110a-c may be adapted for a second medical device.

The “first medical device” may be the same as or different from the “second medical device”. One medical device may require a large number of different dressing pieces, and a variety of dermal areas may thus need protection. The respective guiding lines of the first and second sets of guiding lines can provide a plurality of different dressing pieces adapted for different dermal areas, but still configured to be used with the same medical device.

The at least one marking may comprise a fold marking 111 indicating that the dressing should be folded prior to cutting.

The fold marking 111 may be formed by a print pattern, a weld pattern, a compressed pattern, an embossed pattern, a material deposition pattern, or combinations thereof.

Typically, the fold marking 111 is a printed marking.

The fold marking 111 is not limited to a specific shape, but any marking that guides the caregiver to fold the dressing may be utilized. The fold marking 111 may be arranged in a central position on the folding line 107.

In FIGS. 1A and 1B, the fold marking 111 comprises a curved arrow, which serves to emphasize that the dressing should be folded. Accordingly, the staff or caregivers are directed to folding the dressing prior to cutting the dressing into pieces.

The at least one marking (106a-c, 110a-c, 111) is typically printed marking(s) formed by a colored ink.

Any colored ink may be used as long as it remains permanently adhered to the first release liner. Preferably, a flexographic ink is used. The ink may be water based or solvent based; i.e. the ink may comprise an organic solvent, such as an alcohol, ester etc. that can dissolve the pigment, resin and potentially other additives.

The markings may be printed on the first release liner by any conventional printing technique known in the art, including, but not limited to a gravure printing, a flexographic printing, an offset printing, an ink jet printing and the like. Preferably, a flexographic printing technique is utilized.

The markings may alternatively, or additionally, be embossed. The embossed markings may facilitate cutting of the dressing due to the difference in tactility.

In embodiments where the markings are embossed, the embossed markings may be provided by conventional embossing techniques using e.g. thermal bond, ultrasonic bond and/or pressure. For example, the first release liner may be arranged between two rolls, wherein one of the rolls is engraved with the visual pattern and the other is flat.

As illustrated in FIGS. 1A and 1B, the first portion 108 of the first release liner 103 has a texture, color or shade that differs from the texture, color or shade of the second portion 109 of the first release liner 103.

The difference in texture, color and/or shade of the first, and the second portion, respectively, may serve to emphasize that the dressing should be folded, and/or to emphasize that the respective portion yield different dressing shapes tailored for different facial areas and different types of medical devices (depending on which portion the caregiver chooses to cut).

The difference in color or shade may also be regarded as visually appealing to the viewer.

In FIGS. 1A-C, the first portion 108 is white or transparent and the second portion 109 is colored.

Accordingly, a distinct difference between the portions is provided.

The colored second portion 109 of the first release liner 103 and the at least one printed marking are formed by the same colored ink, wherein the shade of the colored second portion 109 is lighter than the shade of the at least one printed marking.

By utilizing the same colored ink for printing both the second portion and the markings, the manufacturing of the first release liner is significantly improved. By varying the contrast and shade of the color of the ink, a distinct and visually appealing contrast is created between the printed markings and the colored second portion. When the markings comprise a first and/or a second set of guiding lines, the shade of the printed guiding lines may also vary between the guiding lines of the first and second sets of printed guiding lines.

Furthermore, in embodiments where the fold marking 111 is a printed marking, the same ink may be utilized as that used for printing the first and/or second sets of guiding lines.

The difference in color and/or shade of the first, and the second portion distinguishes the respective sets of guiding lines from one another, and the caregiver may choose to cut from the colored or the transparent/white portion depending on the desired dressing shapes and medical device to be utilized. The difference in color and/or shade also emphasizes that the dressing should be folded prior to cutting.

Preferably, the ink has a color that contrast with the color of the first release liner. For example, the color of the ink may be grey, black, purple, pink, blue, green, red and any shades thereof.

Preferably, the color of the ink is PMS 254 (Pantone Matching System).

The difference in the shade between the second portion and the printed guiding lines may be from 40 to 80%, e.g. from 50 to 70%.

The “difference in shade” may e.g. be a difference in opacity of the colored ink, a difference in the degree of saturation of the colored ink or a difference in raster density of the colored ink.

The first release liner 103 may have a surface tension that is higher than the surface tension of the colored ink.

This allows for an improved adherence of the colored ink to the first release liner. The surface tension of the first release liner may be increased by subjecting the first release liner to corona treatment.

The lateral (x) extension typically defines the width of the first release liner, and the longitudinal (y) extension typically defines the length of the first release liner. The width of the second release liner 104 corresponds to the width of the first release liner 101, and wherein the length of the second release liner 104 corresponds to from 20 to 60%, preferably from 30 to 40% of the length of the first release liner 103.

Accordingly, in embodiments where the first release liner 103 comprises a folding line and first and/or second sets of guiding lines, the second release liner extends in a transverse direction with respect to the folding line.

This arrangement is beneficial, since after the dressing pieces have been cut into their respective shaped dressing pieces, the majority of the dressing pieces will comprise at least a portion of the second, smaller release liner. Accordingly, the removal of the first release liner overlying each of the cut-out dressing pieces will be facilitated.

The first release liner 103 may be defined by a first longitudinal edge 112a and a second longitudinal edge 112b; the first 112a and the second 112b longitudinal edges extending in parallel to each other in the lateral (x) direction, and a first lateral edge 113a and a second lateral edge 113b; the first 113a and the second 113b lateral edges extending in parallel to each other in the longitudinal (y) direction.

The dressings in FIGS. 1A-1C are square shaped. Consequently, the lateral (x) extension and the longitudinal (y) extension are the same. Also, each lateral and longitudinal edge have the same length. If the dressing has a rectangular shape, the lateral (x) extension is larger than the longitudinal (y) extension (or vice versa). The dressing of the present disclosure is by no means limited to a specific shape, but any shape may be conceivable.

The “longitudinal (y) extension” corresponds to the length direction of the first release liner (or the dressing).

The “lateral (x) extension” corresponds to the width direction of the first release liner (or the dressing).

A suitable width and/or length of a medical dressing of the present disclosure is in the range of from 8 to 25 cm, preferably from 10 to 20 cm, and most preferably from 12 to 15 cm.

The width of the first release liner is measured from the first lateral edge 113a to the second lateral edge 113b. The length of the first release liner is measured from the first longitudinal edge 112a to the second longitudinal edge 112b.

As illustrated in FIG. 1B, the second release liner 104 extends between the first 113a and the second 113b lateral edges and is arranged to cover at least a portion of the first 106a, second 106b and third 106c guiding lines of the first set of guiding lines, as well as the first guiding line 110a of the second set of guiding lines. Accordingly, all of the dressing pieces formed when cutting through these guiding lines will comprise a part of the second release liner. Accordingly, removal of the first release liner 103 from the dressing pieces is facilitated. The dressing piece formed by the third guiding line 110c of the second set of guiding lines will only be covered by the first release liner 103.

The first release liner 103 has at least two opposing peripheral edges (112a-b, 113a-b) and the at least one marking extends from one peripheral edge (112a-b, 113a-b) to another peripheral edge (112a-b, 113a-b).

With reference to FIGS. 1A and 1B, the first set of guiding lines 106a-c of the first portion 108 comprises at least a first guiding line 106a extending from a first point 114 on the first longitudinal edge 112a in a curved path to an opposing first point 115 on the second longitudinal edge 112b.

The curved path forms a convex curve between the first point 114 on the first longitudinal edge 112a and the opposing first point 115 on the second longitudinal edge 112b. The convex curve is arranged to bulge towards the folding line 107.

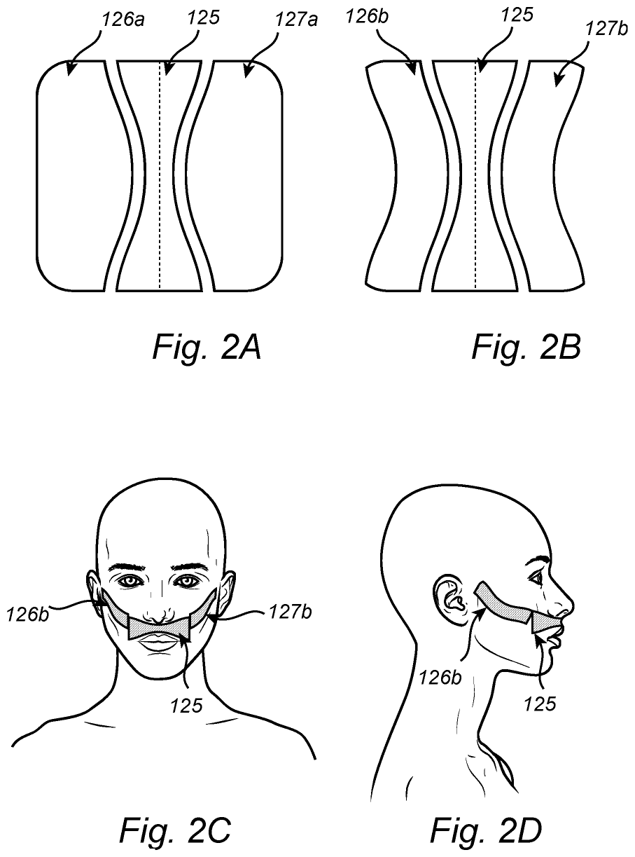

When cutting the first guiding line, the dressing pieces (125, 126a, 127a) illustrated in FIG. 2A are obtained. Accordingly, a centrally disposed, substantially hourglass shaped dressing piece 125 and two respective edge pieces (126a, 127a) are provided.

The hourglass shaped dressing 125 has a narrow waste portion which improves the conformability of the dressing when applied under the nose of a patient.

The central point of the convex curve formed by the first guiding line 106a is preferably arranged at a distance of from 3 to 12 mm, e.g from 5 to 10 mm from the folding line 107. This is to yield an hourglass shaped dressing having a waste portion particularly suited for placement under the nose.

The first set of guiding lines 106a-c of the first portion 108 may comprise a second guiding line 106b; the second guiding line 106b extending from a second point 116 on the first longitudinal edge 112a to an opposing second point 117 on the second longitudinal edge 112b; the second guiding line 106b being arranged at a distance from, and generally following the same curved path as the first guiding line 106a.

The curved path forms a convex curve between the second point 116 on the first longitudinal edge 112a to the opposing second point 117 on the second longitudinal edge 112b. The convex curve is arranged to bulge towards the folding line 107 and generally follows the same curved path as the first guiding line 106a.

When cutting the second guiding line 106b, the dressing shapes (126b, 127b) illustrated in FIG. 2B are formed. These shapes resemble the shapes of FIG. 2A, but are trimmed at the edges.

The dressing shapes formed by cutting the first guiding line 106a, and optionally the second guiding line 106b are adapted to fit under e.g. an oxygen tube, and serve to protect the cheeks and the area beneath the nose as illustrated in FIGS. 2C and 2D.

The hourglass shaped dressing 125 may be arranged beneath the nose, and the respective edge piece (126a-b, 127a-b) may be arranged adjacent to the hourglass shaped dressing 125 and extend across the cheeks towards the ears (as illustrated in FIG. 2D).

As illustrated in FIG. 1A, the first set of guiding lines 106a-c of the first portion 108 comprises a third guiding line 106c extending from a first point 118 on the first lateral edge 113a in a curved path to a third point 119 on the second longitudinal edge 112b.

The third point 119 on the second longitudinal edge 112b is arranged between the first point 115 from which the first guiding line extends, and the second point 117 from which the second guiding line 106b extends.

As can be seen in FIG. 1A, the third guiding line forms a slightly convex curve between the respective end-points (118 and 119). The third guiding line 106c intersects the second guiding line 106b.

The first release liner may be divided by a lateral (x) center line into an upper portion and a lower portion. The first point 118, from which the third guiding line 106 extends is arranged in the upper portion of the first release liner.

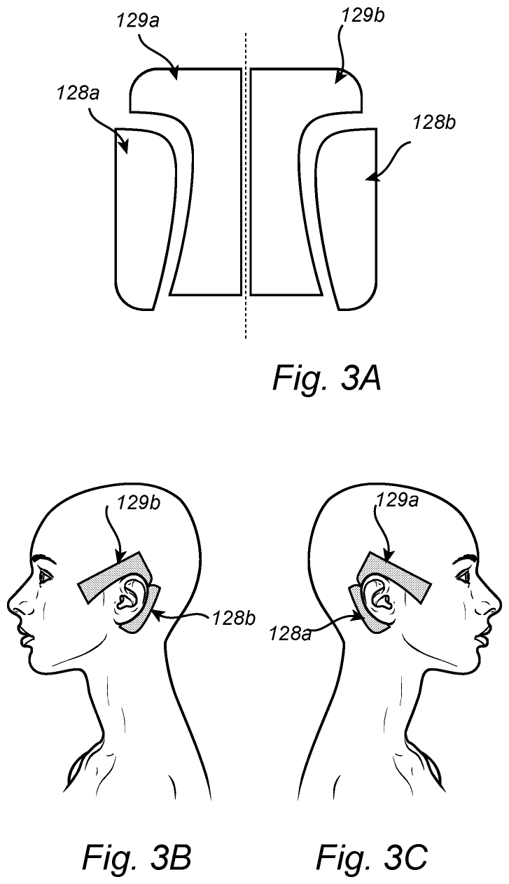

When cutting the third guiding line 106c, the dressing pieces 128a-b in FIG. 3A are formed. These shapes may e.g. be utilized to protect the area behind the ears (see FIGS. 3B and 3C).

The folding line 107 may be a guiding line indicating that the folding line 107 is to be cut

As illustrated in FIG. 3A, when cutting through the folding line 107, the shaped dressing pieces (128a-b, 129a-b) are thereby obtained.

The dressing pieces resulting from cutting the folding line 107 may be arranged to cover the upper area of the ear and extend towards the cheek and/or forehead of the patient (see FIGS. 3B and 3C).

The dressing pieces obtained by means of cutting the first set of guiding lines and as illustrated in FIGS. 2A-D and 3A-3C offer full protection of the skin when e.g. an oxygen tubing is to be applied to a patient.

As illustrated in FIGS. 1A and 1B, the second set of guiding lines 110a-c of the second portion 109 comprises at least a first guiding line 110a extending from a first point 120 on the second lateral edge 113b, and a second guiding line 110b extending from a second point 121 on the second lateral edge 113b; the first 110a and the second 110b guiding lines being connected at a connecting point 122 in the second portion 109 such that the first 110a and the second 110b guiding lines form the shape of a substantially curved triangle.

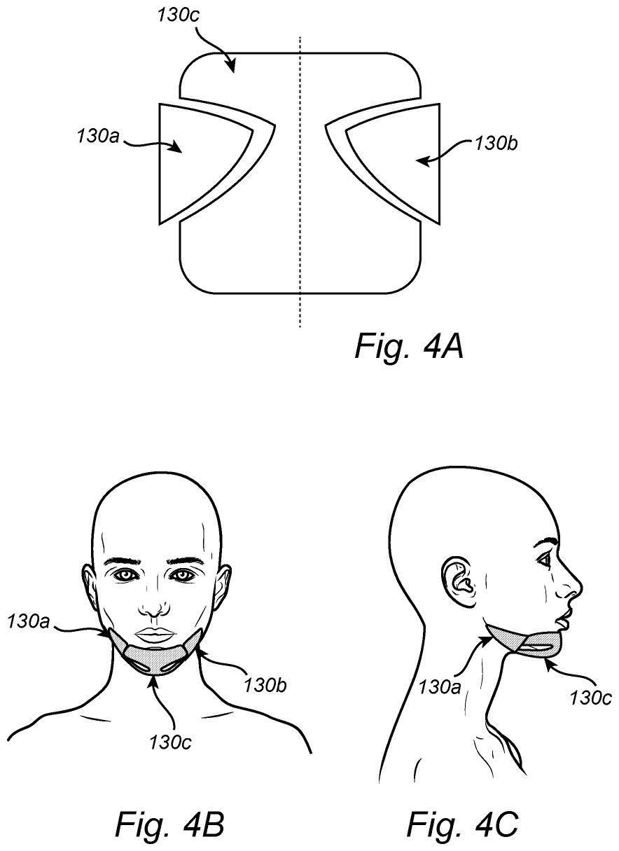

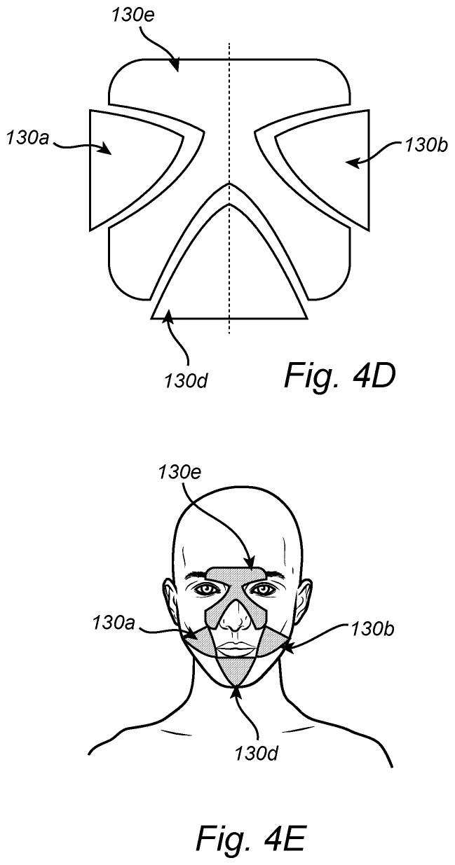

Accordingly, three dressing pieces (130a-c) that are suitable to dress the cheeks or the chin of the patient are obtained (see FIGS. 4B and 4C). Such an arrangement is beneficial for use in conjunction with a cervical collar or a tracheostomy tubing.

The first release liner may be divided by a lateral (x) center line into an upper portion and a lower portion. The first point 120 on the second lateral edge 113b, from which the first guiding line 110a extends, is arranged in the upper portion. The second point 121 on the second lateral edge 113b, from which the second guiding line 110b extends is arranged in the lower portion.

The connecting point 122 connecting the first 110a and second 110b guiding lines in the second portion 109 is preferably arranged in the upper portion of the first release liner. The connecting point 122 may be provided at a distance of from 6 to 13 mm, e.g. from 8 to 10 mm from the folding line 107. This is to improve the fit to the bridge of the nose of a patient.

The second set of guiding lines 110a-c of the second portion 109 may comprise a third guiding line 110c extending in a curved path from a first point 123 on the folding line 107 to a first point 124 on the second longitudinal edge 112b.

The first release liner may be divided by a lateral (x) center line into an upper portion and a lower portion. The first point 123 of the folding line 107 is arranged in the lower portion, i.e. below a central point of the folding line. If the first release liner comprises a centrally disposed fold marking 111, the first point 123 of the folding line is arranged adjacent to the fold marking, but does not coincide therewith.

Accordingly, when the dressing is cut in the folded configuration, a substantially curved triangle is provided, similar in shape to that defined by the first and the second guiding lines, explained hereinbefore.

The dressing pieces (130a-b, 130d-e) illustrated in FIG. 4D are thereby obtained. The centrally disposed dressing piece 130e has the general shape of a horizontal letter K and is larger than the remaining dressing pieces (130a-b, 130d). The dressing pieces may be used to dress a patient in accordance with FIG. 4E. In FIG. 4E, the dressing piece 130e is arranged to cover a portion of the forehead and extending partly over the nose bridge and the areas adjacent to the nose. The substantially triangular dressing pieces (130a, 130b, 130d) are arranged to cover the cheeks and the chin in the area circumventing the mouth of a patient.

Such an arrangement is suitable for use in conjunction with a CPAP mask.

As illustrated in FIGS. 1A-C, the respective guiding lines are associated with scissor markings 131, to further emphasize that the guiding lines should be cut.

In another aspect, the present disclosure relates to a kit comprising a medical dressing as described hereinbefore and at least one medical device

Terms, definitions and embodiments of all aspects of the present disclosure apply mutatis mutandis to the other aspects of the present disclosure.

Even though the present disclosure has been described with reference to specific exemplifying embodiments thereof, many different alterations, modifications and the like will become apparent for those skilled in the art.

Variations to the disclosed embodiments can be understood and effected by the skilled addressee in practicing the present disclosure, from a study of the drawings, the disclosure, and the appended claims. Furthermore, in the claims, the word “comprising” does not exclude other elements or steps, and the indefinite article “a” or “an” does not exclude a plurality.

1. A medical dressing comprising a backing layer, an adhesive skin contact layer and a first release liner detachably attached to said adhesive skin contact layer; said dressing further comprising a second release liner arranged between said adhesive skin contact layer and said first release liner, wherein the surface area of said second release liner is smaller than the surface area of said first release liner, characterized in that said first release liner is co-extensive with said backing layer and said adhesive skin contact layer and in that said first release liner comprises at least one marking.

2. The medical dressing according to claim 1, wherein said at least one marking indicates how to cut, fold, use or apply said dressing.

3. The medical dressing according to claim 1, wherein said first release liner is defined by a central portion and an edge portion surrounding said central portion, wherein said central portion constitutes from 70 to 95% of the surface area of said first release liner, wherein said at least one marking is arranged in at least said edge portion of said first release liner.

4. The medical dressing according to claim 1, wherein said at least one marking is formed by a print pattern, a weld pattern, a compressed pattern, an embossed pattern, a material deposition pattern, or combinations thereof.

5. The medical dressing according to claim 1, wherein said first release liner and said second release liner are formed from different materials.

6. The medical dressing according to claim 1, wherein said first release liner comprises a first polymeric film, and wherein said second release liner comprises a second polymeric film, wherein said first polymeric film is different from said second polymeric film.

7. The medical dressing according to claim 6, wherein said first and/or said second polymeric film is selected from a polyethylene film, a polypropylene film and a polyurethane film.

8. The medical dressing according to claim 6, wherein said first polymeric film is a polyethylene film and wherein said second polymeric film is a polypropylene film.

9. The medical dressing according to claim 1, wherein said dressing comprises a pad arranged between said backing layer and said adhesive skin contact layer.

10. The medical dressing according to claim 1, wherein said first release liner has a lateral (x) extension and a longitudinal (y) extension, wherein said first release liner comprises a longitudinal center line dividing said first release liner into a first portion and a second portion; said first and said second portion being symmetric about said longitudinal center line, wherein said first portion comprises a first set of markings, and wherein said second portion comprises a second set of markings, and wherein said first set of markings is different from said second set of markings.

11. The medical dressing according to claim 10, wherein said first set of markings comprises a first set of guiding lines indicating how to cut the dressing into a first set of dressing pieces, and wherein said second set of markings comprises a second set of guiding lines indicating how to cut the dressing into a second set of dressing pieces.

12. The medical dressing according to claim 10, wherein said longitudinal center line is a folding line, and wherein said dressing can be arranged in a first, flat configuration, and in a second, folded configuration about said folding line, wherein said first set of guiding lines indicate how to cut said dressing in said folded configuration, and wherein said second set of guiding lines indicate how to cut said dressing in said folded configuration.

13. The medical dressing according to claim 10, wherein the dressing pieces of said first set of dressing pieces are adapted for a first medical device and wherein the dressing pieces of said second set of dressing pieces are adapted for a second medical device.

14. The medical dressing according to claim 11, wherein said at least one marking comprises a fold marking indicating that the dressing should be folded prior to cutting.

15. The medical dressing according to claim 1, wherein said at least one marking is a printed marking formed by a colored ink.

16. The medical dressing according to claim 10, wherein said first portion of said first release liner has a texture, color or shade that differs from the texture, color or shade of said second portion of said first release liner.

17. (canceled)

18. (canceled)

19. The medical dressing according to claim 15, wherein said first release liner has a surface tension that is higher than the surface tension of said colored ink.

20. The medical dressing according to claim 1, wherein said first release liner has lateral (x) extension defining the width of the first release liner, and a longitudinal (y) extension defining the length of the first release liner, wherein the width of said second release liner corresponds to the width of said first release liner, and wherein the length of said second release liner corresponds to from 20 to 60% of the length of said first release liner.

21. The medical dressing according to claim 1, wherein said first release liner has at least two opposing peripheral edges and wherein said at least one marking extends from one peripheral edge to another peripheral edge.

22. A kit comprising a medical dressing according to claim 1 and at least one medical device.
